(19) United States
(12) Patent Application Publication
(10) Pub. No.: US 2016/0053302 A1
(43) Pub. Date: Feb. 25, 2016

(54) METHOD FOR VISUAL IDENTIFICATION OF PCR SOLUTIONS FOR ACCURATE REACTION SETUP

(71) Applicant: Roche Molecular Systems, Inc., Pleasanton, CA (US)

(72) Inventors: Amar Gupta, Danville, CA (US); Nancy Schoenbrunner, Charlestown, MA (US); Kevin Janssen, Philadelphia, PA (US); Victoria Brophy, Martinez, CA (US)

(21) Appl. No.: 14/688,114

(22) Filed: Apr. 16, 2015

Related U.S. Application Data
(60) Provisional application No. 61/982,456, filed on Apr. 22, 2014.

(51) Int. Cl. C12Q 1/68

(52) U.S. Cl. CPC C12Q 1/6806

(57) ABSTRACT

The present invention provides for methods and compositions that use visible dyes for the identification of reagents and solutions that are used to perform PCR assays.

### CROSS REFERENCE TO RELATED INVENTION

This application claims the benefit of priority of U.S. Provisional Patent Application Ser. No. 61/982,456, filed Apr. 22, 2014, which is incorporated herein by reference in its entirety.

### FIELD OF THE INVENTION

The present invention is related to the field of nucleic acid amplification by the polymerase chain reaction (PCR) assay. In particular, the invention pertains to use of visible dyes for the identification of reagents that are used to perform PCR assays.

### BACKGROUND OF THE INVENTION

PCR is a powerful technique for amplifying DNA or RNA that can be used for a wide variety of purposes. The myriad of applications of PCR include its usage for the diagnosis of viral or bacterial genes and the identification of genetic mutations. Currently, PCR assays can be performed in real-time, homogenous formats, e.g. the TaqMan® assay, and using instruments that can test up to four nucleic acid sequences simultaneously. In a typical TaqMan® assay, target nucleic acids are detected by use of quenched fluorescent probes that are cleaved during PCR amplification, resulting in an increase in the fluorescence signals. However, in order to control for variability that may occur during PCR and also for optimization of the sensitivity for a given PCR assay, different enzymes, metal cofactors and concentrations of the constituents required for performing PCR are often used. In practice, many of these constituents are pre-mixed into a single solution, called a mastermix, on a per-assay basis. Because of this, assays with the same fluorophores but with different constituents may be performed side-by side (e.g. in adjacent wells on a multiwell plate) and without prior identification, the reactions would be indistinguishable, meaning that a positive fluorescence signal may be misinterpreted as a positive result for the wrong target nucleic acid. Presently, there is an absence of methodologies to combat the difficulties in assuring that a correct mastermix is used in a PCR assay for a given target nucleic acid. This problem is particularly critical in a clinical laboratory setting to ensure that no user error has occurred. Although the prevalence of these errors should be low, their occurrence may result in the generation of false positive or false negative results which are extremely important to attenuate.

### SUMMARY OF THE INVENTION

The present invention addresses the need to create certainty in the proper preparation of reagents and mastermix solutions that are used in PCR assays and lead to a higher confidence in the data generated from such PCR assays. In one aspect the invention provides for a method of preparing a plurality of reaction mixtures for performing polymerase chain reaction (PCR) amplification of a plurality of target nucleic acids, comprising providing a first mastermix solution comprising at least one substance required for performing PCR amplification of a first target nucleic acid, and further comprising a first visible dye; providing a second mastermix solution comprising at least one substance required for performing PCR amplification of a second target nucleic acid, and further comprising a second visible dye that is different in color from said first visible dye; adding said first mastermix solution to a first reaction mixture to perform PCR amplification of said first target nucleic acid; and adding said second mastermix solution to a second reaction mixture to perform PCR amplification of said second target nucleic acid; wherein amplification is detected by measurement of fluorescence, and wherein said first visible dye and said second visible dye do not inhibit PCR amplification and are present at concentrations whereby fluorescence emission is inhibited by no greater than 50%.

In another aspect, the invention provides for a method of preparing a plurality of reaction mixtures for performing polymerase chain reaction (PCR) amplification of a plurality of target nucleic acids, comprising providing a first mastermix solution comprising at least one substance required for performing PCR amplification of a first target nucleic acid, and further comprising a visible dye that is present at a first predetermined concentration; providing a second mastermix solution comprising at least one substance required for performing PCR amplification of a second target nucleic acid, and further comprising said visible dye that is present at a second predetermined concentration, wherein the presence of said visible dye at said first predetermined concentration can be visually distinguished from the presence of said visible dye at said second predetermined concentration; adding said first mastermix solution to a first reaction mixture to perform PCR amplification of said first target nucleic acid; and adding said second mastermix solution to a second reaction mixture to perform PCR amplification of said second target nucleic acid; wherein amplification is detected by measurement of fluorescence, and wherein said visible dye does not inhibit PCR amplification and is present at a concentration whereby fluorescence emission is inhibited by no greater than 50%.

In one embodiment of the present invention, the visible dye is selected from the group consisting of a phthalocyanine dye, a diazine dye, wherein said diazine dye is not Neutral Red, a thiazine dye, Malachite Green, Fast Corinth V, and Crystal Violet. In some embodiments, the phthalocyanine dye is Alcian Blue, the diazine dye is Azocarmine G, and the thiazine dye is New Methylene Blue.

In yet another aspect, the invention provides for a kit for performing polymerase chain reaction (PCR) amplification of a plurality of target nucleic acids, comprising a plurality of mastermix solutions wherein each one mastermix solution from the plurality of mastermix solutions comprises at least one substance required for performing PCR amplification of a specific target nucleic acid, and further comprises a visible dye selected from the group consisting of a phthalocyanine dye, a diazine dye wherein said diazine dye is not Neutral Red, a thiazine dye, Malachite Green, Fast Corinth V, and Crystal Violet. In some embodiments, the phthalocyanine dye is Alcian Blue, the diazine dye is Azocarmine G, and the thiazine dye is New Methylene Blue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G show the chemical structures and absorbance wavelengths of several visible dyes: Malachite Green (FIG. 1A), Fast Corinth V (FIG. 1B), New Methylene Blue (FIG. 1C), Azocarmine G (FIG. 1D), Crystal Violet (FIG. 1E), Neutral Red (FIG. 1F), Alcian Blue (FIG. 1G).

FIG. 2 shows a photograph of the 96-well plate used in the experiment described in Example 1. R=Neutral Red, G=Malachite Green, Y=Fast Corinth V, P=Crystal Violet, NCC=no dye. Rows 1, 2, 3 contain different concentrations of each of the dyes.

FIG. 3 shows the PCR growth curves generated using a JA270-labelled probe that detects amplification of the T790M EGFR template DNA in the presence of Crystal Violet (purple lines), Neutral Red (red lines), Malachite Green (green lines), Fast Corinth V (yellow lines), and no dye control NCC (black lines).

FIGS. 4A-D show the PCR growth curves generated using a FAM-labelled probe that detects amplification of the T790M EGFR template DNA in the presence of (FIG. 4A) Crystal Violet (purple lines), (FIG. 4B) Neutral Red (red lines), (FIG. 4C) Malachite Green (green lines), (FIG. 4D) Fast Corinth V (yellow lines). Black lines represent no dye controls.

FIGS. 5A-B show the PCR growth curves generated using a FAM-labelled probe that detects amplification of the T790M EGFR template DNA in the presence of (FIG. 5A) Malachite Green at 7.5 mg/L and 15 mg/L, (orange lines), (FIG. 5B) Fast Corinth V at 15 mg/L and 30 mg/L (purple lines). Black lines represent no dye controls.

### DETAILED DESCRIPTION OF THE INVENTION

### Definitions

The term “sample” as used herein includes a specimen or culture (e.g., microbiological cultures) that includes nucleic acids. The term “sample” is also meant to include both biological and environmental samples. A sample may include a specimen of synthetic origin. Biological samples include whole blood, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), biopsy sample, urine, feces, sputum, saliva, nasal mucous, prostate fluid, semen, lymphatic fluid, bile, tears, sweat, breast milk, breast fluid, embryonic cells and fetal cells. In a preferred embodiment, the biological sample is blood, and more preferably plasma. As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The terms “target” or “target nucleic acid” as used herein are intended to mean any molecule whose presence is to be detected or measured or whose function, interactions or properties are to be studied. Therefore, a target includes essentially any molecule for which a detectable probe (e.g., oligonucleotide probe) or assay exists, or can be produced by one skilled in the art. For example, a target may be a biomolecule, such as a nucleic acid molecule, a polypeptide, a lipid, or a carbohydrate, that is capable of binding with or otherwise coming in contact with a detectable probe (e.g., an antibody), wherein the detectable probe also comprises nucleic acids capable of being detected by methods of the invention. As used herein, “detectable probe” refers to any molecule or agent capable of hybridizing or annealing to a target biomolecule of interest and allows for the specific detection of the target biomolecule as described herein. In one aspect of the invention, the target is a nucleic acid, and the detectable probe is an oligonucleotide. The terms “nucleic acid” and “nucleic acid molecule” may be used interchangeably throughout the disclosure. The terms refer to oligonucleotides, oligos, polynucleotides, deoxyribonucleotide (DNA), genomic DNA, mitochondrial DNA (mtDNA), complementary DNA (cDNA), bacterial DNA, viral DNA, viral RNA, RNA, message RNA (mRNA), transfer RNA (tRNA), ribosomal RNA (rRNA), siRNA, catalytic RNA, clones, plasmids, M13, P1, cosmid, bacteria artificial chromosome (BAC), yeast artificial chromosome (YAC), amplified nucleic acid, amplicon, PCR product and other types of amplified nucleic acid, RNA/DNA hybrids and polyamide nucleic acids (PNAs), all of which can be in either single- or double-stranded form, and unless otherwise limited, would encompass known analogs of natural nucleotides that can function in a similar manner as naturally occurring nucleotides and combinations and/or mixtures thereof. Thus, the term “nucleotides” refers to both naturally-occurring and modified/nonnaturally-occurring nucleotides, including nucleoside tri, di, and monophosphates as well as monophosphate monomers present within polynucleic acid or oligonucleotide. A nucleotide may also be a ribo; 2′-deoxy; 2′,3′-deoxy as well as a vast array of other nucleotide mimics that are well-known in the art. Mimics include chain-terminating nucleotides, such as 3′-O-methyl, halogenated base or sugar substitutions; alternative sugar structures including nonsugar, alkyl ring structures; alternative bases including inosine; deaza-modified; chi, and psi, linker-modified; mass label-modified; phosphodiester modifications or replacements including phosphorothioate, methylphosphonate, boranophosphate, amide, ester, ether; and a basic or complete internucleotide replacements, including cleavage linkages such a photocleavable nitrophenyl moieties.

The presence or absence of a target can be measured quantitatively or qualitatively. Targets can come in a variety of different forms including, for example, simple or complex mixtures, or in substantially purified forms. For example, a target can be part of a sample that contains other components or can be the sole or major component of the sample. Therefore, a target can be a component of a whole cell or tissue, a cell or tissue extract, a fractionated lysate thereof or a substantially purified molecule. Also a target can have either a known or unknown sequence or structure.

The term “amplification reaction” refers to any in vitro means for multiplying the copies of a target sequence of nucleic acid.

“Amplifying” refers to a step of submitting a solution to conditions sufficient to allow for amplification. Components of an amplification reaction may include, but are not limited to, e.g., primers, a polynucleotide template, polymerase, nucleotides, dNTPs and the like. The term “amplifying” typically refers to an “exponential” increase in target nucleic acid. However, “amplifying” as used herein can also refer to linear increases in the numbers of a select target sequence of nucleic acid, but is different than a one-time, single primer extension step.

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990. PCR as used in the present invention also includes reverse-transcription PCR (RT-PCR) (Joyce (2002) “Quantitative RT-PCR. A review of current methodologies” Methods Mol Biol. 193:83-92) whereby the amplicon generation is by reverse transcribing an RNA nucleic acid target and amplifying by a polymerase chain reaction.

“Oligonucleotide” as used herein refers to linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target nucleic acid. Usually monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. Where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill.

As used herein “oligonucleotide primer”, or simply “primer”, refers to a polynucleotide sequence that hybridizes to a sequence on a target nucleic acid template and facilitates the detection of an oligonucleotide probe. In amplification embodiments of the invention, an oligonucleotide primer serves as a point of initiation of nucleic acid synthesis. In non-amplification embodiments, an oligonucleotide primer may be used to create a structure that is capable of being cleaved by a cleavage agent. Primers can be of a variety of lengths and are often less than 50 nucleotides in length, for example 12-25 nucleotides, in length. The length and sequences of primers for use in PCR can be designed based on principles known to those of skill in the art.

The term “oligonucleotide probe” as used herein refers to a polynucleotide sequence capable of hybridizing or annealing to a target nucleic acid of interest and allows for the specific detection of the target nucleic acid.

The term “reagent solution” is any solution containing at least one reagent needed or used for PCR purposes. Most typical ingredients are polymerase, nucleotide, primer, ions, magnesium, salts, pH buffering agents, deoxynucleotide triphosphates (dNTPs), probe, fluorescent dye (may be attached to probe), nucleic acid binding agent, a nucleic acid template. The reagent may also be other polymerase reaction additive, which has an influence on the polymerase reaction or its monitoring.

The term “mastermix” refers to a mixture of all or most of the ingredients or factors necessary for PCR to occur, and in some cases, all except for the template and primers which are sample and amplicon specific. Commercially available mastermixes are usually concentrated solutions. A mastermix may contain all the reagents common to multiple samples, but it may also be constructed for one sample only. Using mastermixes helps to reduce pipetting errors and variations between samples due to differences between pipetted volumes.

The term “visible dye” refers to any substance that is capable of being homogeneously mixed or dissolved within a solution and capable of giving the solution a perceivable color. “Color” herein means any detectable spectral response of a solution to white light in the visual range. “Different colors” mean that the colors are distinguishable, preferably by the naked eye, but at least with spectral determination means.

Different colors may have maximum peaks in their absorbance spectrum separated by at least 30 nm. Preferably, the different colors are selected from the groups of red, yellow, blue, cyan, magenta and visually distinguishable combinations and shades thereof, such as green, orange and violet. Therefore, a visible dye according to the present invention will have an absorbance wavelength between 300 and 700 nm, preferably between 350 and 650 nm. For each visible dye, there will be a concentration range whereby the visual intensity of the dye can be distinguished by the naked eye. Some dyes are visible to the naked eye at concentrations as low as 0.25 milligrams per liter (mg/L) in solution while other dyes may require concentrations as high as 50 mg/L to be clearly visible. Therefore, a visible dye according to the present invention will be present at a concentration range between 0.25 mg/L and 50 mg/L.

Each visible dye that is used to practice the present invention should not significantly interfere with the measurement of fluorescence meaning that the presence of the visible dye at the concentration range used in the reaction mixture should not inhibit the detection of the fluorescent signal that is generated during the PCR amplification reaction to the extent that the presence or absence of the amplified target nucleic acid cannot be determined with confidence. In practice, the visible dye should be used at a concentration whereby the fluorescence emission in the presence of the visible dye is not lower than 50% of the fluorescence emission that is detected when the visible dye is absent. Therefore, a visible dye according to the present invention is present at a concentration whereby fluorescence emission is inhibited by no greater than 50%. In some embodiments, the visible dye is present at concentrations whereby fluorescence emission is inhibited by no greater than 40%, 30%, 20%, 10% or 5%, or is inhibited by less than 5%.

A “diazine dye” refers to any of a class of organic chemical compounds containing a benzene ring in which two of the carbon atoms have been replaced by nitrogen atoms. Exemplary diazine dyes include an azocarmine dye, a phenazine dye, an oxazine dye, and diethylsafraninazodimethylaniline chloride (Janus Green B or Diazine Green 5).

A “thiazine dye” refers to any of a class of organic chemical compounds containing a tricyclic aromatic fused ring system, where two of the carbons in the middle ring are replaced by a nitrogen atom and a sulfur atom. Exemplary thiazine dyes include methylene blue, methylene green, thionin, 1,9-dimethylmethylene blue, sym-dimethylthionin, toluidine blue O, new methylene blue, methylene violet bernthsen, azure A, azure B, and azure C.

A “phthalocyanine dye” refers to any of a class of organic chemical compounds containing four pyrrole-like subunits linked to form a 16-membered ring. The pyrrole-like rings within H₂Pc are closely related to isoindole. Both porphyrins and phthalocyanines function as planar tetradentate dianionic ligands that bind metals through four inwardly projecting nitrogen centers. Such complexes are formally derivatives of Pc²⁻, the conjugate base of H₂Pc. Exemplary phthalocyanine dyes include alcian blue, alcec blue, pigment blue 15, and Xerox xpp-TiOPcl.

A “nucleic acid polymerase” refers to an enzyme that catalyzes the incorporation of nucleotides into a nucleic acid. Exemplary nucleic acid polymerases include DNA polymerases, RNA polymerases, terminal transferases, reverse transcriptases, telomerases and the like.

A “thermostable DNA polymerase” refers to a DNA polymerase that is stable (i.e., resists breakdown or denaturation) and retains sufficient catalytic activity when subjected to elevated temperatures for selected periods of time. For example, a thermostable DNA polymerase retains sufficient activity to effect subsequent primer extension reactions, when subjected to elevated temperatures for the time necessary to denature double-stranded nucleic acids. Heating conditions necessary for nucleic acid denaturation are well known in the art and are exemplified in U.S. Pat. Nos. 4,683,202 and 4,683,195. As used herein, a thermostable polymerase is typically suitable for use in a temperature cycling reaction such as the polymerase chain reaction (“PCR”). The examples of thermostable nucleic acid polymerases include Thermus aquaticus Taq DNA polymerase, Thermus sp. Z05 polymerase, Thermus flavus polymerase, Thermotoga maritima polymerases, such as TMA-25 and TMA-30 polymerases, Tth DNA polymerase, and the like.

A “modified” polymerase refers to a polymerase in which at least one monomer differs from the reference sequence, such as a native or wild-type form of the polymerase or another modified form of the polymerase. Exemplary modifications include monomer insertions, deletions, and substitutions. Modified polymerases also include chimeric polymerases that have identifiable component sequences (e.g., structural or functional domains, etc.) derived from two or more parents. Also included within the definition of modified polymerases are those comprising chemical modifications of the reference sequence. The examples of modified polymerases include G46E E678G CS5 DNA polymerase, G46E L329A E678G CS5 DNA polymerase, G46E L329A D640G S671F CS5 DNA polymerase, G46E L329A D640G S671F E678G CS5 DNA polymerase, a G46E E678G CS6 DNA polymerase, Z05 DNA polymerase, ΔZ05 polymerase, ΔZ05-Gold polymerase, ΔZ05R polymerase, E615G Taq DNA polymerase, E678G TMA-25 polymerase, E678G TMA-30 polymerase, and the like.

The term “5′ to 3′ nuclease activity” or “5′-3′ nuclease activity” refers to an activity of a nucleic acid polymerase, typically associated with the nucleic acid strand synthesis, whereby nucleotides are removed from the 5′ end of nucleic acid strand, e.g., E. coli DNA polymerase I has this activity, whereas the Klenow fragment does not. Some enzymes that have 5′ to 3′ nuclease activity are 5′ to 3′ exonucleases. Examples of such 5′ to 3′ exonucleases include: Exonuclease from B. subtilis, Phosphodiesterase from spleen, Lambda exonuclease, Exonuclease II from yeast, Exonuclease V from yeast, and Exonuclease from Neurospora crassa.

The detection of a target nucleic acid utilizing the 5′ to 3′ nuclease activity can be performed by a “TaqMan®” or “5′-nuclease assay”, as described in U.S. Pat. Nos. 5,210,015; 5,487,972; and 5,804,375; and Holland et al., 1988, Proc. Natl. Acad. Sci. USA 88:7276-7280, all incorporated by reference herein. In the TaqMan® assay, labeled detection probes that hybridize within the amplified region are present during the amplification reaction. The probes are modified so as to prevent the probes from acting as primers for DNA synthesis. The amplification is performed using a DNA polymerase having 5′ to 3′ nuclease activity. During each synthesis step of the amplification, any probe which hybridizes to the target nucleic acid downstream from the primer being extended is degraded by the 5′ to 3′ nuclease activity of the DNA polymerase. Thus, the synthesis of a new target strand also results in the degradation of a probe, and the accumulation of degradation product provides a measure of the synthesis of target sequences.

Any method suitable for detecting degradation product can be used in a 5′ nuclease assay. Often, the detection probe is labeled with two fluorescent dyes, one of which is capable of quenching the fluorescence of the other dye. The dyes are attached to the probe, typically with the reporter or detector dye attached to the 5′ terminus and the quenching dye attached to an internal site, such that quenching occurs when the probe is in an unhybridized state and such that cleavage of the probe by the 5′ to 3′ nuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated by reference herein, describe alternative methods for detecting the degradation of a probe which occurs concomitant with amplification.

Fluorescent dyes may include dyes that are negatively charged, such as dyes of the fluorescein family, or dyes that are neutral in charge, such as dyes of the rhodamine family, or dyes that are positively charged, such as dyes of the cyanine family. Dyes of the fluorescein family include, e.g., 6-carboxy-fluorescein (FAM), 2′,4, 4′,5′,7, 7′-hexachlorofluorescein (HEX), TET, JOE, NAN and ZOE. Dyes of the rhodamine family include, e.g., Texas Red, ROX, R110, R6G, and TAMRA or the rhodamine derivative JA270 (see, U.S. Pat. No. 6,184,379, issued Feb. 6, 2001, to Josel et al.). FAM, HEX, TET, JOE, NAN, ZOE, ROX, R110, R6G, and TAMRA are commercially available from, e.g., Perkin-Elmer, Inc. (Wellesley, Mass., USA), and Texas Red is commercially available from, e.g., Molecular Probes, Inc. (Eugene, Oreg.). Dyes of the cyanine family include, e.g., Cy2, Cy3, Cy5, Cy 5.5 and Cy7, and are commercially available from, e.g., Amersham Biosciences Corp. (Piscataway, N.J., USA).

A 5′ nuclease assay for the detection of a target nucleic acid can employ any polymerase that has a 5′ to 3′ nuclease activity. Thus, in some embodiments, the polymerases with 5′-nuclease activity are thermostable and thermoactive nucleic acid polymerases. Such thermostable polymerases include, but are not limited to, native and recombinant forms of polymerases from a variety of species of the eubacterial genera Thermus, Thermatoga, and Thermosipho, as well as chimeric forms thereof. For example, Thermus species polymerases that can be used in the methods of the invention include Thermus aquaticus (Taq) DNA polymerase, Thermus thermophilus (Tth) DNA polymerase, Thermus species Z05 (Z05) DNA polymerase, Thermus species sps17 (sps17), and Thermus species Z05 (e.g., described in U.S. Pat. Nos. 5,405,774; 5,352,600; 5,079,352; 4,889,818; 5,466,591; 5,618,711; 5,674,738, and 5,795,762. Thermatoga polymerases that can be used in the methods of the invention include, for example, Thermatoga maritima DNA polymerase and Thermatoga neapolitana DNA polymerase, while an example of a Thermosipho polymerase that can be used is Thermosipho africanus DNA polymerase. The sequences of Thermatoga maritima and Thermosipho africanus DNA polymerases are published in International Patent Application No. PCT/US91/07035 with Publication No. WO 92/06200. The sequence of Thermatoga neapolitana may be found in International Patent Publication No. WO 97/09451.

In the 5′ nuclease assay, the amplification detection is typically concurrent with amplification (i.e., “real-time”). In some embodiments the amplification detection is quantitative, and the amplification detection is real-time. In some embodiments, the amplification detection is qualitative (e.g., end-point detection of the presence or absence of a target nucleic acid). In some embodiments, the amplification detection is subsequent to amplification. In some embodiments, the amplification detection is qualitative, and the amplification detection is subsequent to amplification.

The present invention presents an opportunity to internally encode each reaction mixture used in a PCR assay to create certainty in the proper reaction preparation as well as higher confidence in the resulting data. The methods of this invention involve the addition of visual dyes to mastermixes which would allow researchers to visually confirm the set-up for each reaction.

In one aspect, the invention using visual dyes is the addition to mastermixes of at least two specific dyes which must satisfy stringent conditions. The dyes have to be almost completely inert and cause no detrimental effects to PCR or the fluorescence based analyses. Thus, each one dye must be at a concentration high enough to be visible to the researcher, but the dye must not inhibit hybridization, denaturation, polymerase function, fluorescence excitation, or fluorescence emission to an extent that is pernicious to the PCR assay, especially a real-time PCR assay in which amplification is detected by the measurement of fluorescence. These dyes must also be thermostable enough to satisfy each of the conditions in the presence of heat in a thermocycler (e.g. the LightCycler® instrument from Roche Diagnostics). Furthermore, each of the dyes must be disparate or distinguishable in color from each other so that the researcher may easily identify a mastermix by color both before and after reaction set-up.

In another aspect, the invention involves titration of a single dye that allows for the differentiation of mastermixes based on the concentration and therefore visual intensity of the dye. This dye, of course, would have to satisfy all aforementioned conditions (e.g. thermostable, not inhibit fluorescence) at all concentration used. An additional advantage of this aspect of the invention is the benefit of plausible usage by researchers who are colorblind. Because the differentiation would be based on visual intensity rather than by pigment (wavelength), this method can be performed by any researcher with vision.

The following examples and figures are provided to aid the understanding of the present invention, the true scope of which is set forth in the appended claims. It is understood that modifications can be made in the procedures set forth without departing from the spirit of the invention.

### Examples

### Example 1

### Testing Visible Dyes in PCR with JA270-Labeled Probe

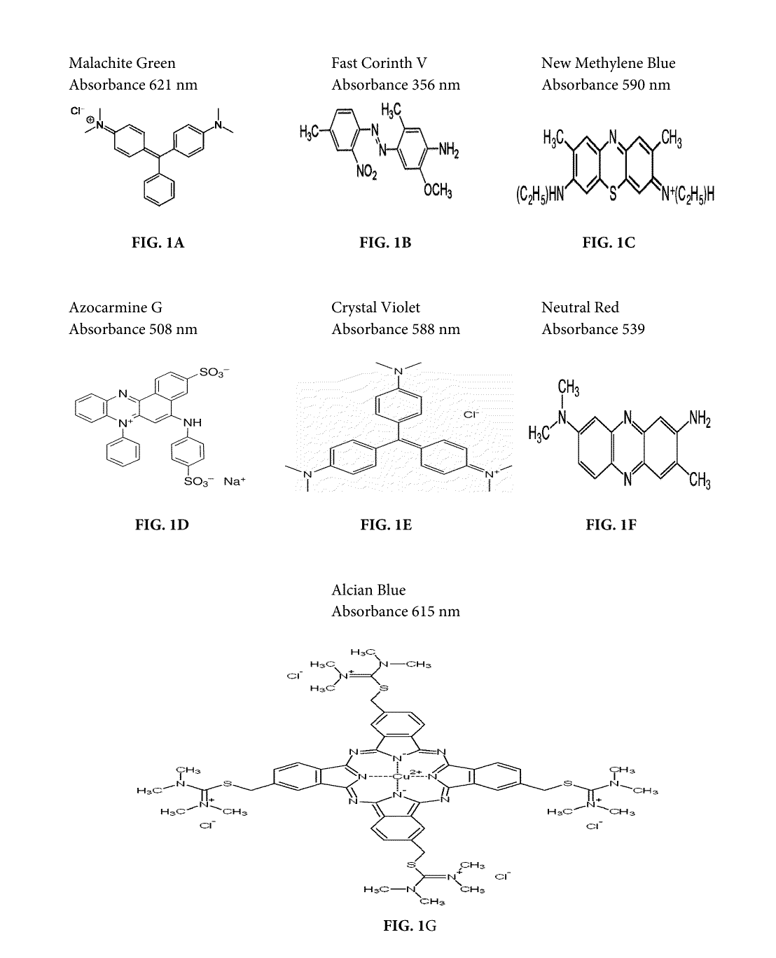

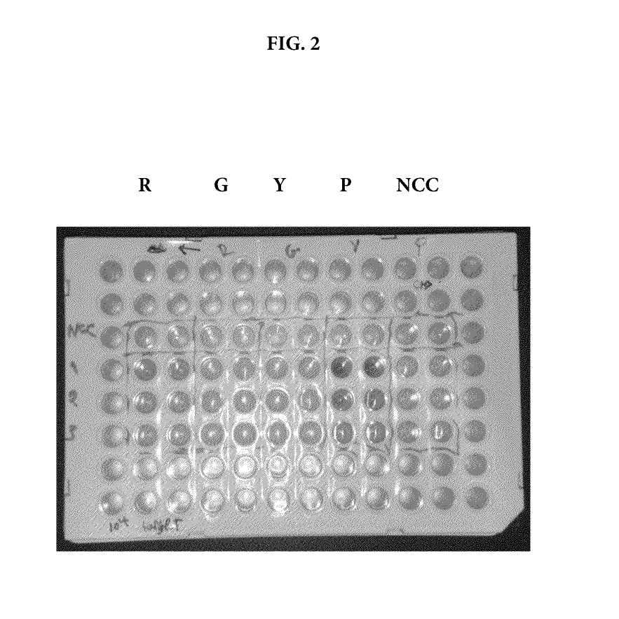

To examine the effects that visible dyes in mastermixes may have on PCR, a PCR assay was performed for the amplification and detection of the T790M mutation (nucleotide change 2369 C->T) of the human Epidermal Growth Factor Receptor (EGFR) gene present in 1×10⁴ copies on a template plasmid. FIG. 1 shows the chemical structures and absorbance wavelengths of the dyes that were tested: Neutral Red (orange), Malachite Green (green), Fast Corinth V (yellow) and Crystal Violet (purple). PCR reaction mixtures were prepared on a 96-well plate with the following final concentrations: 50 mM Tris-HCl (pH 8.0), 80-100 mM potassium chloride, 200 μM each dATP, dCTP and dGTP, 400 μM dUTP, 250 nM of each primer, 200 nM TaqMan® probe labelled with JA270 fluorescent dye (650 nm emission wavelength), target DNA (10,000 copies of EGFR plasmid), 20 nM DNA polymerase (with 5′ nuclease activity), 0.1 mM EDTA, 2.5 mM magnesium acetate. Each dye was tested at three different concentrations, that ranged from 1.0, 1.9 and 3.9 mg/L for Neutral Red and Malachite Green, 1.9, 3.9 and 7.8 mg/L for Fast Corinth V, and 0.5, 1.0 and 1.9 mg/L for Crystal Violet. A photograph of the 96-well plate is shown on FIG. 2 which demonstrates that the dyes are clearly visible in the wells.

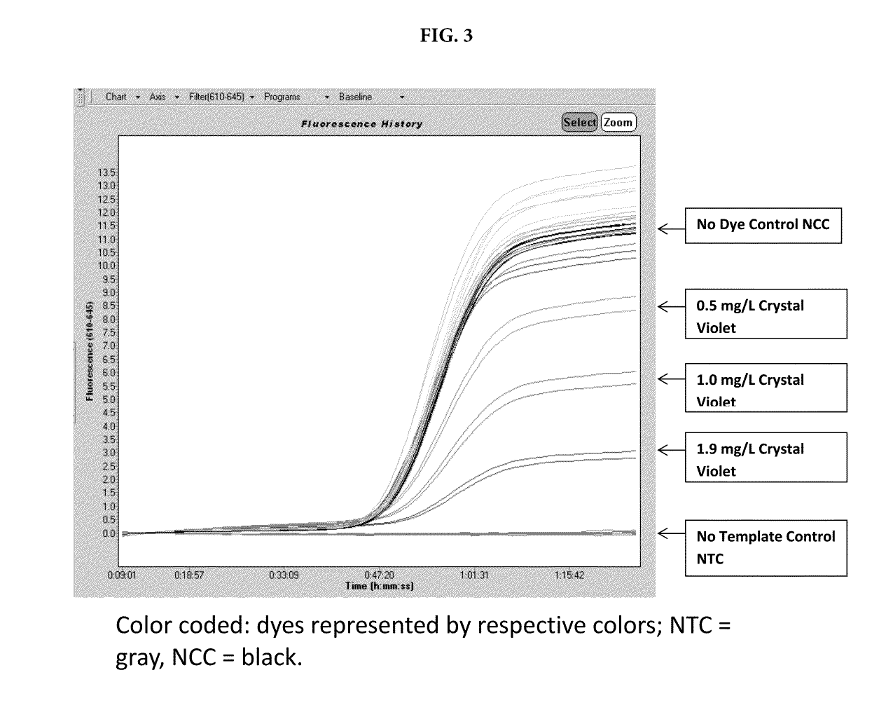

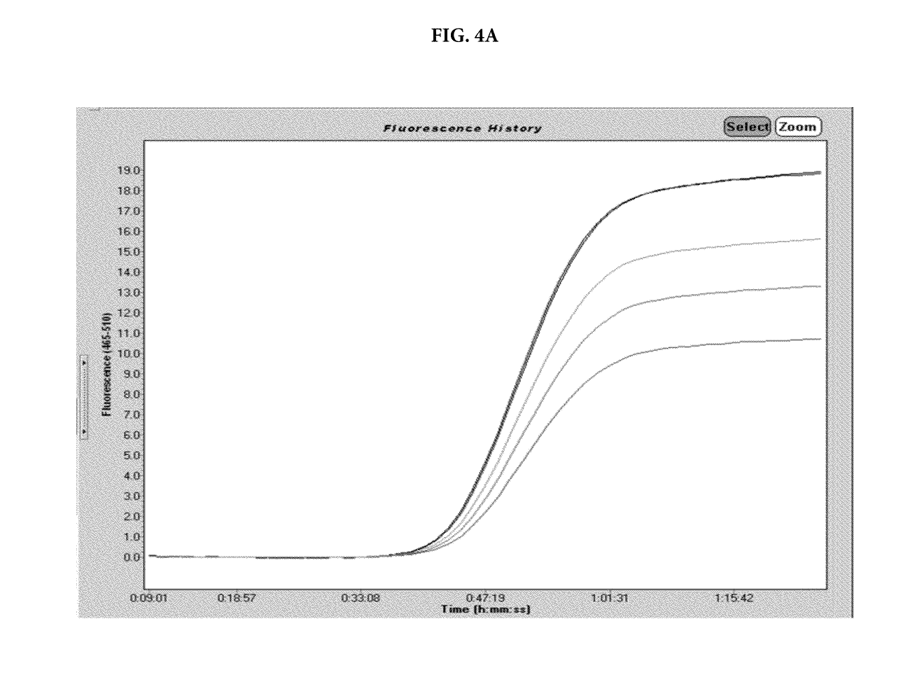

Amplification and analysis was done using the Roche LightCycler® 480 instrument (Roche Applied Science, Indianapolis, Ind.) The following temperature profile was used: 95° C. for 1 minute (or 2 cycles of 95° C. (10 seconds) to 62° C. (25 seconds) followed by cycling from 92° C. (10 seconds) to 62° C. (25-30 seconds) 99 times. FIG. 3 shows the PCR growth curves generated in mastermixes containing Crystal Violet (purple lines), Neutral Red (red lines), Malachite Green (green lines), and Fast Corinth V (yellow lines), each represented as the increase in fluorescent signal over cycle time. At the highest concentration of Crystal Violet, the fluorescent signal was only 25% of the fluorescent signal from the no dye control (NCC wells), whereas in the two lower concentrations of Crystal Violet (0.5 mg/L and 1.0 mg/L), the fluorescent signals were approximately 50% and 75%, respectively, that of the no dye control. Also, when tested at 0.25 mg/L concentration, Crystal Violet exhibited no detectable decrease in fluorescence (data not shown). In contrast, fluorescence signals did not decrease significantly in the PCR growth curves generated from mastermixes that contained Neutral Red, Malachite Green, or Fast Corinth V (FIG. 3) which would indicate the preferential utilities of these dyes in mastermixes for PCR assays that detect a JA270 signal.

### Example 2

### Testing Visible Dyes in PCR with FAM-Labeled Probe

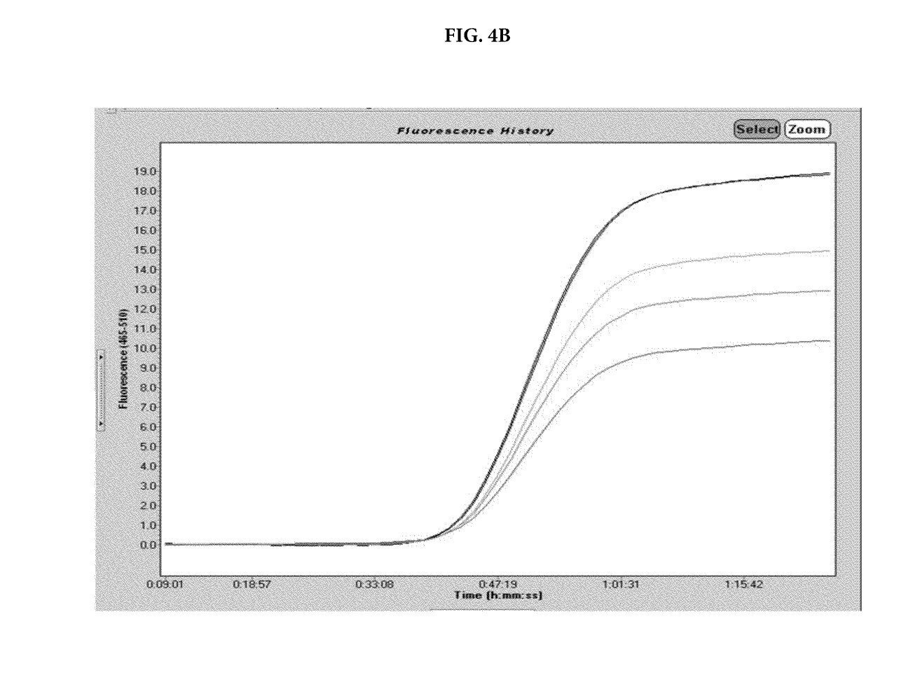

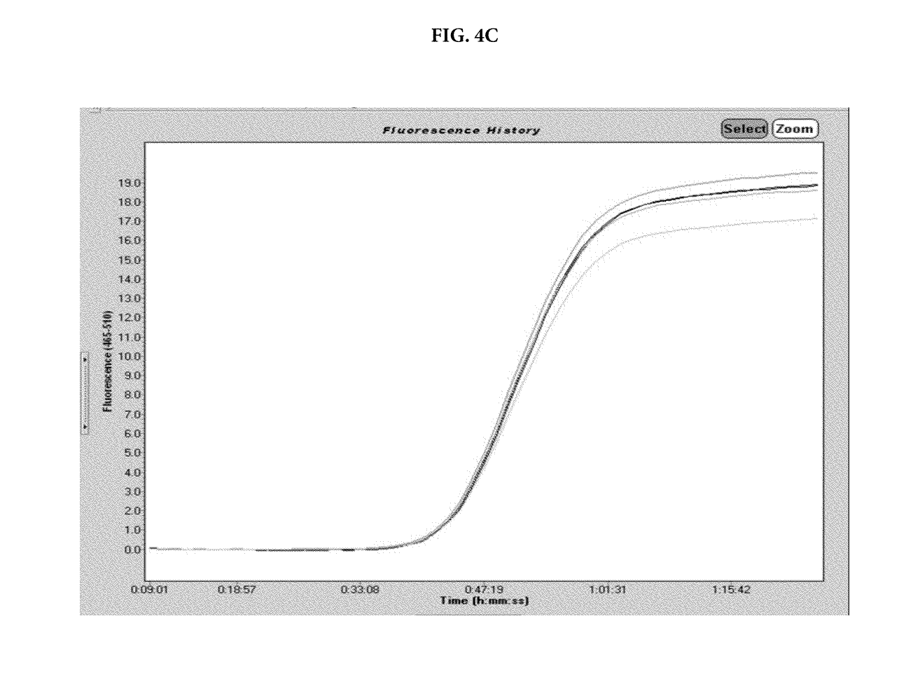

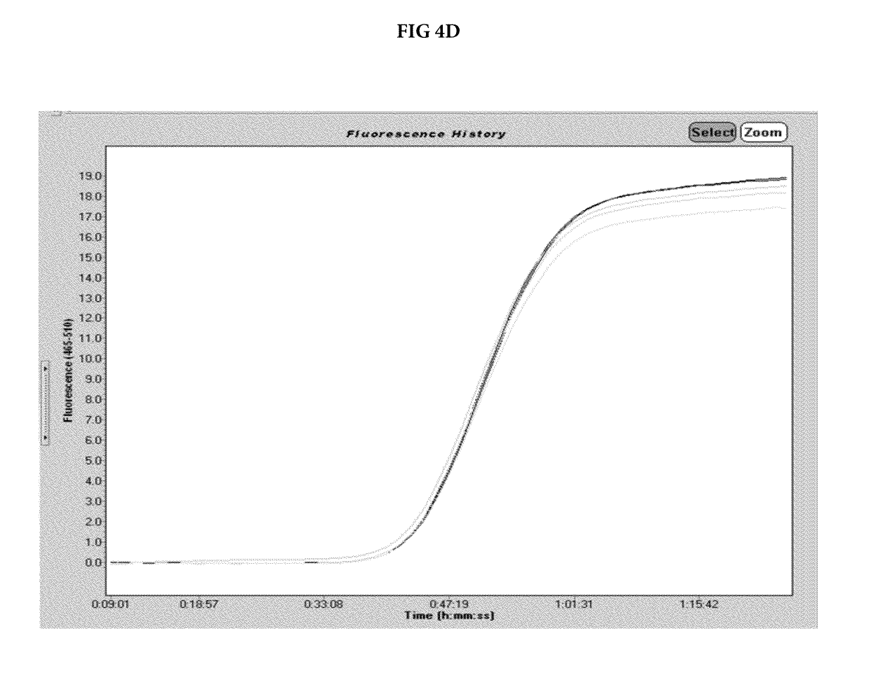

A PCR assay was performed using the identical reagents and conditions as those described in Example 1 except for the use of 200 nM TaqMan® probe labelled with the FAM fluorescent dye. This time fluorescence signals were observed to be decreased in PCR assays performed in the presence of both Crystal Violet and Neutral Red, but even at their highest concentrations, the fluorescence signals were all greater than 50% of the fluorescence signals of the no dye controls (FIG. 4A, 4B). In comparison Malachite Green and Fast Corinth V showed no decrease in FAM signal in their respective PCR growth curves (FIG. 4C, 4D). Other visible dyes that were tested with the FAM-labelled probe that did not decrease fluorescence by over 50% were New Methylene Blue at 2.1 mg/L and 4.2 mg/L, Azocarmine G at 1.0 mg/L, and Alcian Blue at 3.9 mg/L (data not shown).

### Example 3

### Testing Visible Dyes at Higher Concentrations

A PCR assay was performed using the conditions as described in Example 2 except this time, Malachite

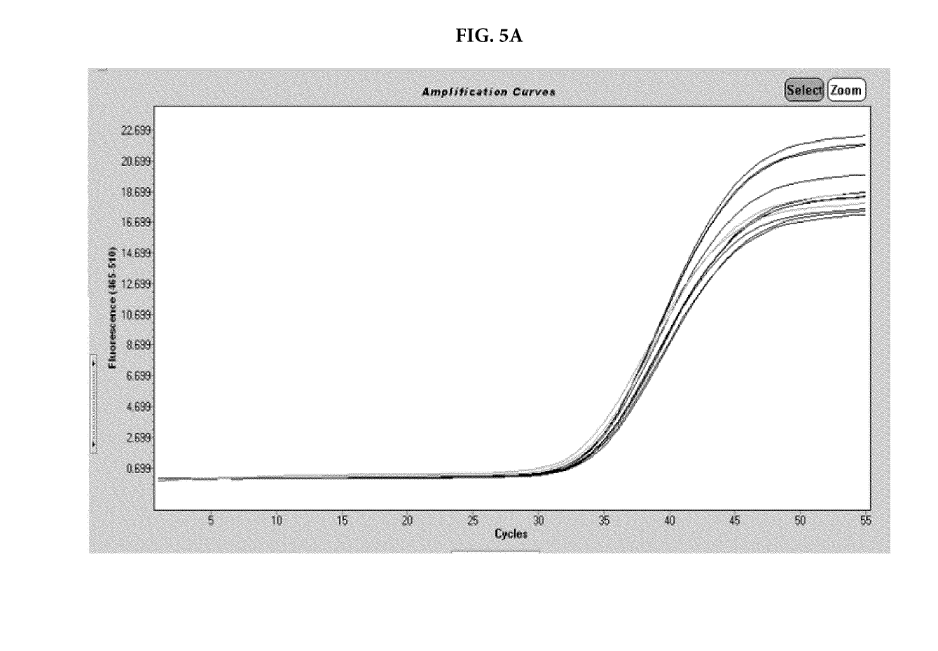

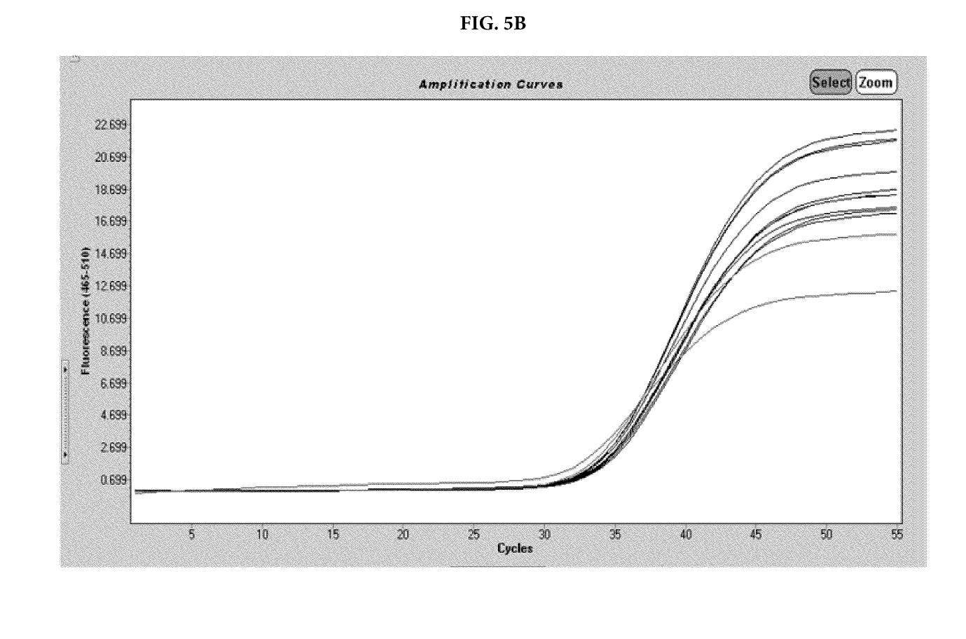

Green was tested at concentrations of 7.5 mg/L and 15.0 mg/L and Fast Corinth V was tested at concentrations of 15.0 mg/L and 30 mg/L. As seen in FIG. 5A, no significant decrease in fluorescence signal is detected with Malachite Green even at 15.0 mg/L. Further experiments showed that when tested using a concentration up to 50 mg/L, Malachite Green still did not decrease fluorescence signal to an intensity that was less than 50% that of the no dye control (data not shown). For Fast Corinth V, the fluorescence signal at 15.0 mg/L was almost 80% and at 30 mg/L was approximately 55% the fluorescence signal of the no dye control (FIG. 5B).

1. A method of preparing a plurality of reaction mixtures for performing polymerase chain reaction (PCR) amplification of a plurality of target nucleic acids, comprising:
providing a first mastermix solution comprising at least one substance required for performing PCR amplification of a first target nucleic acid, and further comprising a first visible dye;
providing a second mastermix solution comprising at least one substance required for performing PCR amplification of a second target nucleic acid, and further comprising a second visible dye that is different in color from said first visible dye;
adding said first mastermix solution to a first reaction mixture to perform PCR amplification of said first target nucleic acid; and
adding said second mastermix solution to a second reaction mixture to perform PCR amplification of said second target nucleic acid;
wherein amplification is detected by measurement of fluorescence, and wherein said first visible dye and said second visible dye do not inhibit PCR amplification and are present at concentrations whereby fluorescence emission is inhibited by no greater than 50%.

2. The method of claim 1 wherein each of said first visible dye and said second visible dye is present at a concentration range between 0.25 mg/L and 50 mg/L.

3. The method of claim 1 wherein said first visible dye and said second visible dye is selected from the group consisting of a phthalocyanine dye, a diazine dye wherein said diazine dye is not Neutral Red, a thiazine dye, Malachite Green, Fast Corinth V and Crystal Violet.

4. The method of claim 3 wherein said phthalocyanine dye is Alcian Blue, said diazine dye is Azocarmine G, and said thiazine dye is New Methylene Blue.

5. A method of preparing a plurality of reaction mixtures for performing polymerase chain reaction (PCR) amplification of a plurality of target nucleic acids, comprising:
providing a first mastermix solution comprising at least one substance required for performing PCR amplification of a first target nucleic acid, and further comprising a visible dye that is present at a first predetermined concentration;
providing a second mastermix solution comprising at least one substance required for performing PCR amplification of a second target nucleic acid, and further comprising said visible dye that is present at a second predetermined concentration, wherein the presence of said visible dye at said first predetermined concentration can be visually distinguished from the presence of said visible dye at said second predetermined concentration;
adding said first mastermix solution to a first reaction mixture to perform PCR amplification of said first target nucleic acid; and
adding said second mastermix solution to a second reaction mixture to perform PCR amplification of said second target nucleic acid;
wherein amplification is detected by measurement of fluorescence, and wherein said visible dye does not inhibit PCR amplification and is present at a concentration whereby fluorescence emission is inhibited by no greater than 50%.

6. The method of claim 5 wherein said visible dye is present at a concentration range between 0.25 mg/L and 50 mg/L.

7. The method of claim 5 wherein said visible dye is selected from the group consisting of a phthalocyanine dye, a diazine dye wherein said diazine dye is not Neutral Red, a thiazine dye, Malachite Green, Fast Corinth V, and Crystal Violet.

8. The method of claim 7 wherein said phthalocyanine dye is Alcian Blue, said diazine dye is Azocarmine G, and said thiazine dye is New Methylene Blue.

9. A kit for performing polymerase chain reaction (PCR) amplification of a plurality of target nucleic acids, comprising a plurality of mastermix solutions wherein each one mastermix solution from the plurality of mastermix solutions comprises at least one substance required for performing PCR amplification of a specific target nucleic acid, and further comprises a visible dye selected from the group consisting of a phthalocyanine dye, a diazine dye, wherein said diazine dye is not Neutral Red, a thiazine dye, Malachite Green, Fast Corinth V, and Crystal Violet.

10. The kit of claim 9 wherein said phthalocyanine dye is Alcian Blue, said diazine dye is Azocarmine G, and said thiazine dye is New Methylene Blue.
